(19) United States
(12) Patent Application Publication
(10) Pub. No.: US 2008/0289649 A1
(43) Pub. Date: Nov. 27, 2008

(54) APPARATUS AND METHOD FOR SANITIZATION OF PORTABLE DEVICES

(75) Inventor: John Victor Woytkiw, Palm Beach Gardens, FL (US)

Correspondence Address: John Woytkiw, 1061 Vintner Blvd., Palm Beach Gardens, FL, 33410, US

(73) Assignee: Cart Shield USA, LLC, Fort Lauderdale, FL (US)

(21) Appl. No.: 12/126,361

(22) Filed: May 23, 2008

Related U.S. Application Data
(60) Provisional application No. 60/939,942, filed on May 24, 2007.

(51) Int. Cl. B08B 3/10; B08B 13/00

(52) U.S. Cl. 134/1; 134/63; 134/56 R

(57) ABSTRACT

A system and method are provided for sanitizing portable devices (14) without introducing waterborne contaminants and pollution into the environment. A sanitizing system (200) loads, washes, irradiates, and treats with an antimicrobial agent, portable devices (14), such as shopping carts, that are likely microbial transmitters, and recovers substantially all used fluid for recycling or environmentally safe disposal. The sanitizing system (200) can be mobile or stationary to one location, and may be fully or partially automated.

### CROSS-REFERENCE TO RELATED APPLICATIONS

This application is based upon and claims priority from U.S. Provisional Patent Application Ser. No. 60/939,942 filed on May 24, 2007. This application is incorporated herein by reference in its entirety.

### FIELD OF THE INVENTION

The invention relates in general to washing and sanitization systems and more specifically to such systems that minimize or eliminate the discharge of water pollution.

### BACKGROUND OF THE INVENTION

The elimination of water pollution is a well-known problem. Water pollution has long been recognized as a serious problem throughout the world. In 1972, Congress enacted the Federal Water Pollution Control Act Amendments; as amended in 1977, this law became commonly known as the Clean Water Act. The stated purpose of the Clean Water Act is to restore and maintain the chemical, physical, and biological integrity of the Nation's waters. To this end, the Clean Water Act prohibits unlicensed discharges of pollutants into the waters of the United States. However, the requirements of the Clean Water Act and other anti-pollution laws are very exacting, and for many purposes the technology simply does not exist that will allow people to comply.

There is a pressing national need to promote pollution prevention and bring all industries into compliance with pollution laws. Nutrient pollution from anthropogenic sources causes blooms of algae and other aquatic microorganisms that are ecologically harmful. Such blooms can lead to: oxygen depletion in the ecosystem; mass killings of fish and other aquatic life; proliferation of organisms that are pathogenic to humans and wildlife (such as “red tide” and Pfiesteria piscicida); proliferation of organisms that render fish and shellfish unfit for human consumption; the production of algal toxins that kill zooplankton, shellfish, fish, birds, marine mammals, and even humans. Nutrient pollution has been blamed in part for the deaths of coral reefs around the world. Even in less severe cases of nutrient pollution, the proliferation of algae can drive out desirable aquatic species, allowing noxious or invasive species to take over.

Another serious global problem is the transmission of microbially-mediated disease. As antibiotic-resistant strains of pathogenic organisms appear and proliferate among the global population, numerous efforts have been made in health-care facilities (such as hospitals) to ensure that common items than can transmit disease from one person to another are routinely sanitized. However, diseases are commonly transmitted on more mundane items in our everyday world. The common door knob is one well-known example. Less well known, shopping carts carry high concentrations of bacteria. A shopping cart is a perfect transmitter of disease, because it constantly comes into contact with a large number of people, comes into contact with perishable foods and is infrequently washed. One public health expert has opined that the shopping carts in a supermarket are dirtier than the store's restroom; this is hardly surprising, as the toilets are washed every day and shopping carts are rarely washed more often than once per week (many establishments wash the shopping carts only once per month).

Other portable devices have the same problem. Common examples include hand baskets, airport luggage carts, wheelchairs, walkers, meat racks and bakery racks.

Although there has been a long-felt need to develop methods of sanitizing such portable devices, such methods have been elusive. Portable devices like shopping carts are still washed infrequently, due to the labor-intensive nature of the task and due to the danger of water pollution from currently known cleaning methods. At present, most supermarkets clean shopping carts simply by hosing them down with mixtures of water and toxic cleaning agents such as Solusol or phosphate detergents. The runoff from the cleaning process includes food waste, bacteria from the cart and any cleaning chemicals employed. Typically the runoff from the process is allowed to flow into public storm sewers, which are connected to natural waterways, for lack of any practicable means to contain the pollution. Often the combination of cleaning compounds and filth from the shopping carts creates runoff that is considered pollution under Federal and state environmental laws. As a result, in addition to being environmentally destructive, allowing the runoff to enter a storm sewer could constitute an illegal act.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an embodiment of an apparatus in accordance with the present invention;

FIG. 2 illustrates an embodiment of an apparatus in accordance with the present invention;

FIG. 3 illustrates an embodiment of an apparatus in accordance with the present invention;

FIG. 4 illustrates an embodiment of an apparatus in accordance with the present invention;

FIG. 5 illustrates an embodiment of an apparatus in accordance with the present invention;

FIG. 6 illustrates an embodiment of an apparatus in accordance with the present invention.

### DESCRIPTION OF A PREFERRED EMBODIMENT

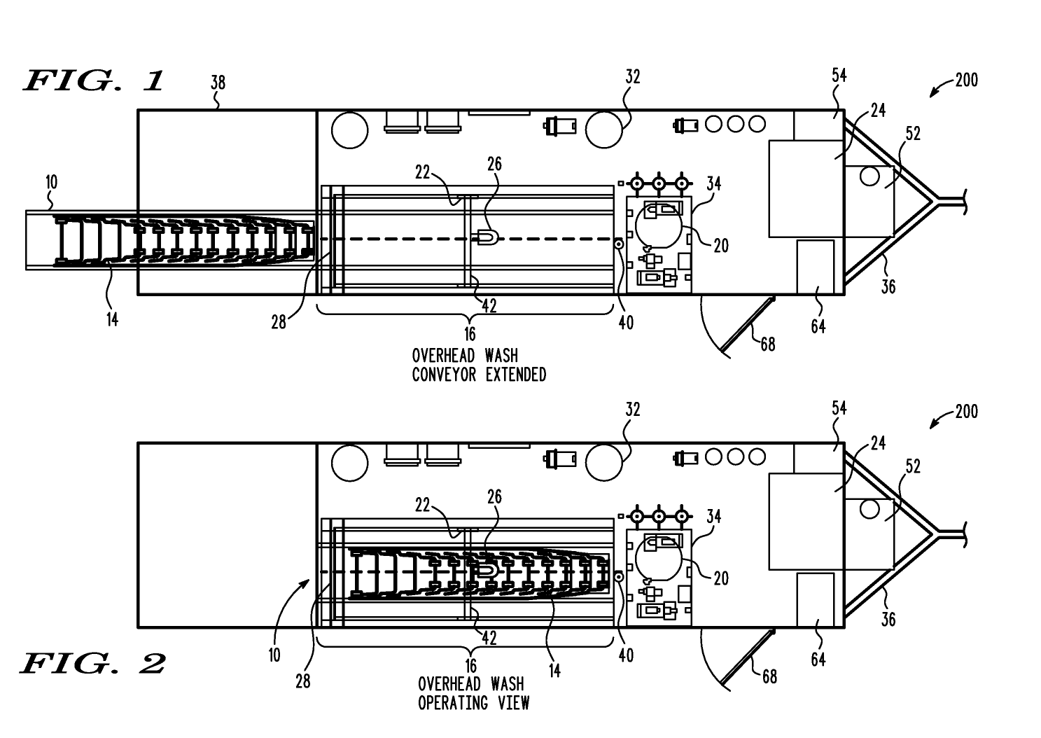

Referring to FIGS. 1, 2, 3, 4, and 5, various embodiments of the present invention are illustrated with respect to a sanitizing system 200. The sanitizing system 200 according to one embodiment includes a loading system 10 that may take many forms. The loading system 10 may be manually operated or it may be automated. Often it will have a piece of equipment, such as a loading gate (not shown in this embodiment) that will prevent at least one portable device 14 (or a plurality of portable devices 14) from shifting during the loading and unloading process. Posts or fasteners may also be present to prevent the at least one portable device 14 from shifting. If the washing area 16 is elevated, the loading system 10 may include a system for raising and lowering the devices; for example, the loading system 10 may be capable of inclination or it may incorporate a lift mechanism to raise and lower the at least one portable device 14 during loading and unloading.

The washing area 16 may take many forms. The washing area 16 may be bounded and/or substantially enclosed such that used washing fluid does not escape into the local environment. The washing area 16 may be enclosed on all sides, including the top. The washing area 16 may include a loading door, that when open allows the loading system 10 to load the device. If the washing area 16 is not completely enclosed, it would preferably be partially enclosed in such a way as to prevent the escape of used washing fluid. The washing area 16 may be positioned near a sump 20 or other passive configuration for collecting used washing fluid.

The washing system 22 of the instant invention may take many forms. Generally, the washing system 22 may expose the portable device 14 to the washing fluid in a manner effective to remove substantially all of the solid or liquid debris (including hydrophobic liquids) from the portable device 14. The washing fluid may contain detergents or other cleaning or antiseptic additives or may be composed only of water or of water with only minor amounts of additives to facilitate recycling. In an embodiment where water is a component of the washing fluid, water may be supplied by a washing fluid reservoir 24 or by an external water supply. A non-aqueous liquid could also be used as the washing fluid. The washing fluid may be delivered to the at least one portable device 14 in the form of a spray, in the form of a stream, through inundation, through submersion, or through other delivery methods.

At least one radiation emitting lamp 26 positioned to expose the portable device 14 to germicidal radiation may take many forms. The radiation emitting lamp 26 may emit ultraviolet radiation, x-ray radiation, gamma radiation, non-ionizing radiation, or other forms of radiation that kill microorganisms, or any combination of the aforementioned radiation. Although x-rays and gamma rays are very effective at killing microorganisms, ultraviolet radiation may be preferred due to the danger of possible exposure of the operator or members of the public to x-rays or gamma rays.

Additionally, an antimicrobial substance applied to the at least one portable device 14, such as via a mist, can be activated when exposed to specific radiation. The specific radiation, according to one embodiment, can be provided by the radiation emitting lamp 26. Moreover, the antimicrobial substance, according one embodiment, bonds to an applied surface of the at least one portable device when the antimicrobial substance is exposed to specific radiation.

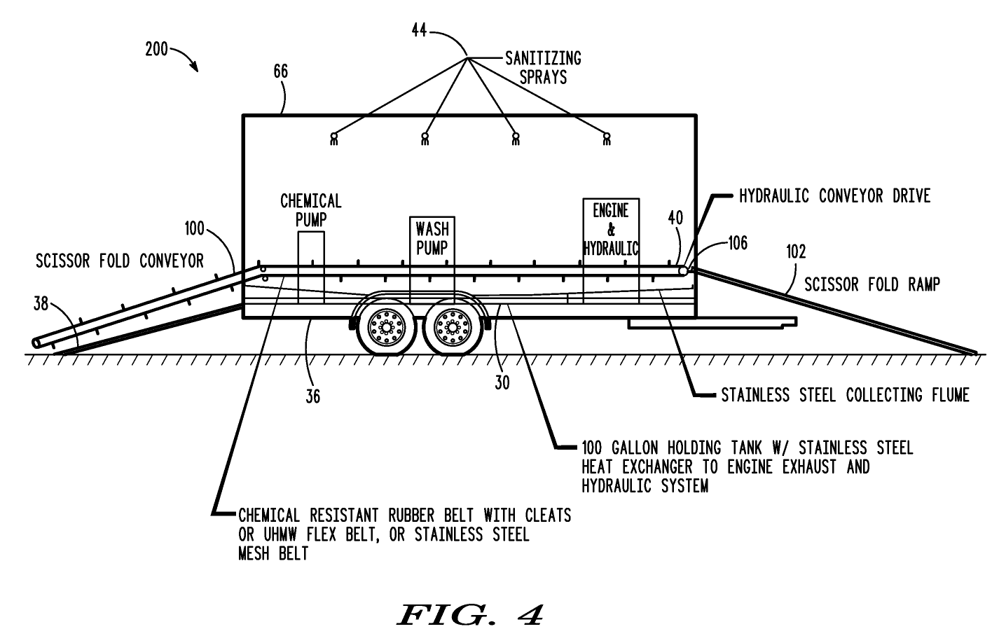

The applicator 28 capable of dispensing an antimicrobial substance can take many forms. The antimicrobial substance may be brought into contact with the portable device 14 in sufficient quantity to provide residual antibacterial effects. The antimicrobial substance can be a disinfectant, a bacteriostat, a microbicide, a virucide or other antimicrobial substance. The applicator 28 may be positioned to allow the portable device 14 to be brought into contact with the antimicrobial substance, and the applicator 28 may be positioned so as to prevent the antimicrobial substance from entering the washing fluid collector 30 (see FIG. 4). The applicator 28 communicates antimicrobial substance with a source of antimicrobial substance 32. The source 32 is generally a tank, reservoir, or other container for liquid or gas.

The washing fluid collector 30 can take many forms, including but not limited to a sump 20, a drain, or a spill tray (not shown). The washing fluid collector 30 may either communicate with a structure for temporary storage of used washing fluid or communicate with a washing fluid recycling system 34.

A washing fluid recycling system 34 may be provided to remove particulates, non-aqueous phase liquids and other unwanted components acquired during the washing process, resulting in purified washing fluid that is fit for reuse in the sanitization system.

The portable devices 14 can be any device with the potential to transmit microorganisms from person to person. These would include devices that are handled by multiple persons, or are exposed to food or biological material, and that will not be damaged by the sanitizing process. Examples of portable devices 14 that can be sanitized by the instant invention include but are not limited to shopping carts, hand baskets, luggage carts, wheel chairs, walkers (a framework designed to support a baby learning to walk or an infirm or disabled person), meat racks, meat carts, baking racks, baking carts and the like.

The present invention may include a source of electrical power. This source can be a generator 64, a battery, or a connection to a local power grid. The generator 64 can be of virtually any type, including those that run on a supply of fossil fuel. The most commonly used fossil fuels for this purpose are diesel and propane, but other fossil fuels may be used. Preferably, the generator 64 would be powered using a type of bio-diesel fuel, and may include features such as heat energy scavenging and recovery that provides a free source to heat water or other liquids used in the cleaning process.

When the generator 64 runs on fossil fuel, the sanitizing system 200 may either include a fuel tank 54 or include a connection to an external fuel source.

As the various systems in the sanitizing system 200 may be substantially automated, a control console may be included to facilitate operation and control of the washing system and components described herein. The control console may include a computer having a processor and/or programmable components that monitor, control, and/or otherwise are coupled to the various systems and components of the sanitizing system 200. The computer can be, for example, a general purpose computer programmed to perform the functions described herein or can be a customized processor based control system. In any case, the control console includes input/output interfaces used to communicate with and control one or more of the various system components described herein. The control console may further include a user interface having one or more visual and/or audio indicators to convey a status or operation of any of the components described herein.

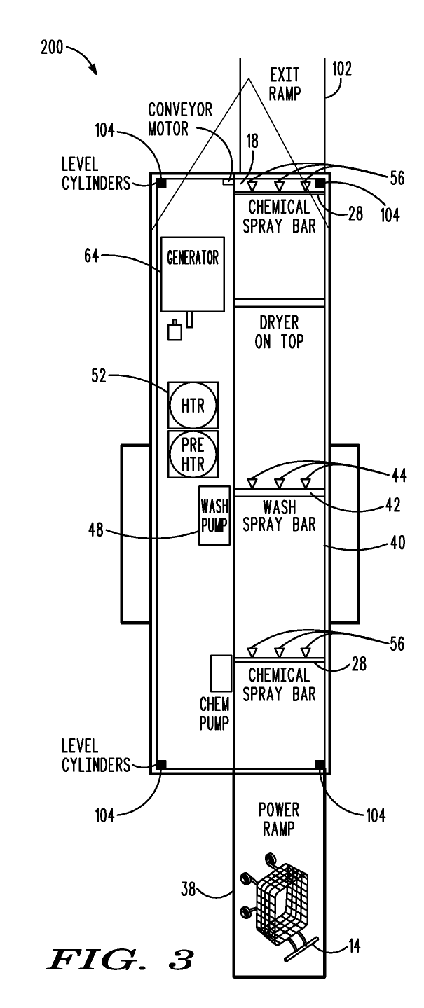

Referring more particularly to FIGS. 3 and 4, the apparatus may be static, or it may be supported by a portable platform 36. Examples of suitable portable platforms 36 include but are not limited to a truck bed or a trailer. Furthermore, the portable platform 36 may include either manual or automatic stabilizing and leveling system 104.

If the apparatus is supported by a portable platform 36, the apparatus may be moved to a site proximate to the portable devices to be sanitized. The portable device 14 will then be sanitized at the site. To transport one or more portable devices 14, an entry ramp with a conveyor 100 and an exit ramp 102 are situated at opposite ends on the platform 36. This allows use of an extremely energy efficient linear feed system that transports the portable devices 14 through the sanitizing system.

Alternatively, the sanitizing system 200 may be quasi-static (e.g., quasi-stationary at a location) or built-in to a retaining area near or in a store or building, and the portable platform 36 may be replaced only when necessary. In such a case, the apparatus can be situated in a maintenance area behind a store or building, and when the sanitizing system 200 needs scheduled servicing or replacement, a new sanitizing system 200 as a modular unit is exchanged for the old one. This sanitizing system 200, utilized in a business model, allows continuous access and use of the sanitizing system 200 while maintaining all the advantages associated with an environmentally friendly implementation.

If the used washing fluid is retained in a used washing fluid collection tank 30 (see FIG. 4), then the sanitizing system 200 as a modular unit may be transported away from the site and the used washing fluid will be disposed of safely in compliance with environmental laws. If the sanitizing system 200 includes a washing fluid recycling system 34 (see FIGS. 1 and 2), then any wastewater produced by the recycling process will be disposed of away from the site safely in compliance with environmental laws.

In one particular embodiment of the present invention, the loading system 10 may include an extendable platform 38. A loading gate is positioned at the loading end of the extendable platform 38, and the loading gate can be raised to prevent the portable devices 14 from falling off the extendable platform 38, or the loading gate can be lowered to allow the portable devices 14 to be moved on and off the extendable platform 38.

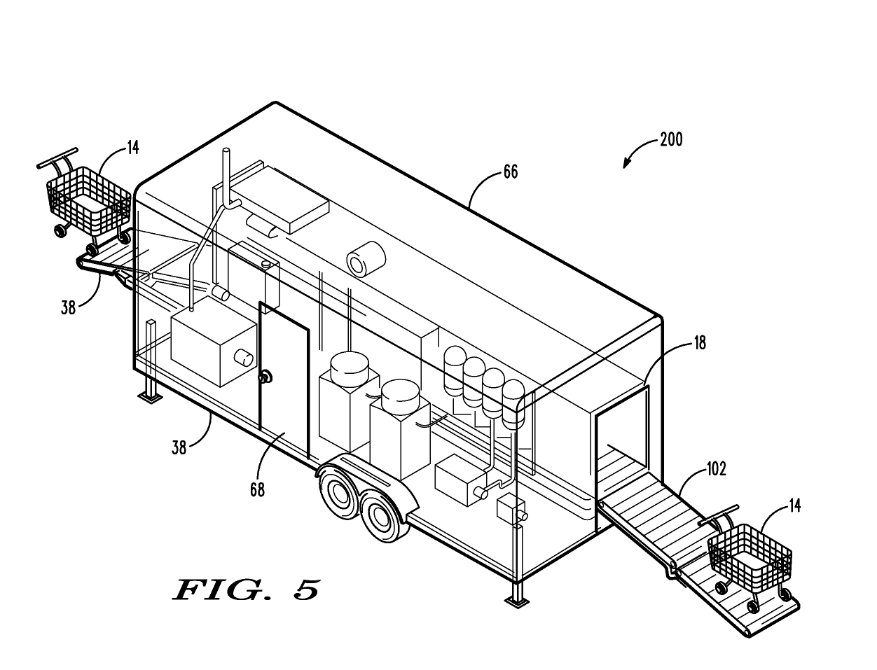

Referring to FIGS. 4 and 5, in its extended position, the extendable platform 38 is in a position in which the portable devices 14 can be easily moved onto the extendable platform 38. In its retracted position, the extendable platform 38 is entirely within the confines of the portable platform 36. The extent to which the extendable platform 38 extends and retracts may be controlled by one or more limit switches. Contemporaneously with the extension or retraction of the extendable platform 38, according to one embodiment, a door at the entrance to the washing bay opens or closes, respectively. The extendable platform 38 may extend and retract at variable speed. In one embodiment where the loading system 10 is automated; the extendable platform 38 may be driven by a conveyor, chain or drive belt system 40. The operator may start the loading process by the touch of a single button, which causes a loading gate to close, the extendable platform 38 to retract, and the door 18 to shut. Optimally, the extendable platform 38 will accommodate 7-10 shopping carts and 3-4 meat or bakery racks or carts.

The washing system 22 may include a movable spray bar 42. The spray bar 42 is capable of movement linearly relative to the at least one portable device 14. The movement of the spray bar 42 is driven by a belt or other mechanical drive 40. Arranged on the spray bar 42 is at least one spray nozzle 44 that emits the washing fluid. A pressure washer may be in fluid communication with the spray nozzles 44 to convey high-pressure water (or other cleaning liquid), according to one embodiment heated to a desired degree, such as approximately 165° F. (80° C.), to the at least one spray nozzle 44, which then directs the (optionally heated) high-pressure water (or other cleaning liquid) in a spray to the at least one portable device 14. The water (or other cleaning liquid) may be provided by an on-board washing fluid reservoir 24 in fluid communication with the pressure washer. The washing fluid reservoir 24 may hold a sufficient volume to allow constant washing of a desired number of portable devices 14, and preferably several cycles or batches of devices. For example, the washing fluid reservoir 24 may hold about 350 gallons. The water, for example, can be delivered from the washing fluid reservoir 24 by a washing fluid supply pump as part of the pressure washer system. The water is preferably heated to a temperature below the boiling point in the washing area 16 (partially determined by the pressure inside the washing area 16). As the hot high-pressure water is sprayed, the spray bar 42 may move back and forth along a track relative to the portable device 14. The operation of the washing system 22 may be substantially automated once activated by the operator, and may occur for a preset period of time, for example, less than five minutes.

The pressure washer may be in communication with a washing fluid heater 52. The washing fluid heater 52 may be fueled by a diesel fuel tank 54, further described below.

The radiation emitting lamp 26 may include an ultraviolet ozone-generating lamp. The radiation emitting lamp 26 may be positioned on the spray bar 42, and the lamp may operate as the spray bar 42 moves back and forth along its track. The radiation emitting lamp 26 can be substantially automated to operate for a pre-determined period of time once activated by the operator. Activation of the washing system 22 by the operator may cause the at least one radiation emitting lamp 26 to automatically assume operation at a set point during and/or after the washing system 22 commences operation. The at least one radiation emitting lamp 26 operates for a predetermined period of time sufficient to provide significant killing of microorganisms on the portable device 14, for example, under five minutes.

The applicator 28 of the present invention may include a low-pressure mister. The low-pressure mister is of the low-volume type, for example, discharging less than 5 gallons per hour. The low discharge rate of the applicator 28 conveys the benefit of low consumption of the antimicrobial substance and less possibility that the antimicrobial substance will escape into the local environment in significant quantity. The applicator 28 communicates with a reservoir containing the antimicrobial substance, and the applicator 28 communicates with an air compressor that facilitates mist formation. The reservoir containing the antimicrobial substance may hold a sufficient volume to allow the application of the antimicrobial substance to at least one load of at least one portable device 14, and preferably several loads. A reservoir, according to one embodiment, may have a capacity of about 20 gallons. The low-pressure mister may be positioned proximate to the exit from the washing area 16 such that portable devices 14 are misted as they are unloaded from the washing area 16.

The washing fluid collector may include a sump 20 in fluid communication with a sump pump and in fluid communication with a washing fluid recycling system 34. The sump pump may start operation automatically when float sensors detect the used washing fluid has reached a predetermined minimum level. The sump pump may also cease operation automatically when float sensors detect the used washing fluid in the sump 20 has fallen to a predetermined level during operation of the sump pump.

The present invention may include a washing fluid recycling system 34 in fluid communication with the on-board washing fluid reservoir 24. The washing fluid recycling system's output per unit time may equal or exceed the output of the washing system 22 per unit time; as a result, the sanitizing system can operate indefinitely with a finite volume of washing fluid, as the washing fluid is recycled during operation. A pump heater may be placed in the washing fluid reservoir 24 to heat and circulate the washing fluid during storage to prevent the washing fluid from freezing in cold climates.

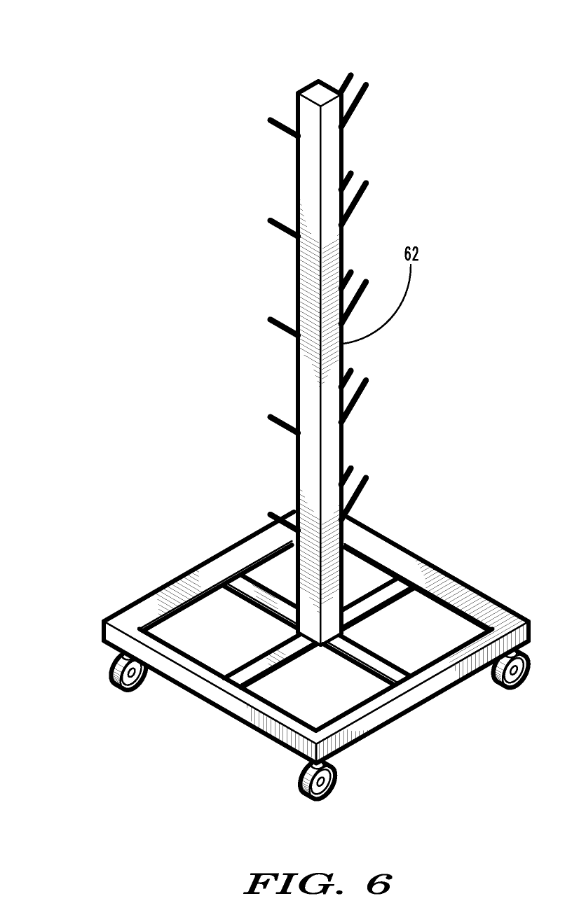

Referring to FIG. 6, as well as FIGS. 1-5, in one embodiment, the portable device 14 is a shopping cart, a hand basket, a meat rack, a bakery rack, a luggage cart, a baby stroller, or any combination thereof. The extendable platform 38 in the loading system 10 may accommodate approximately 7-10 portable devices per load. Alternatively, the extendable platform 38 in the loading system 10 may accommodate 3-4 racks per load, the racks being bakery racks or meat racks or a combination of the two. Hand baskets may be loaded on a basket tree 62. A basket tree 62 is a structure to expose hand baskets to the washing fluid and the germicidal radiation. The basket tree 62 has a base, a vertical trunk resting on the base, and at least one basket hook. The vertical trunk has a lower end and an upper end, and the lower end rests on the base. The basket hook is supported by the vertical trunk. During operation, one or more baskets may be suspended from the basket hooks. The base may be on rollers that allow the basket tree 62 to be easily moved onto and off of the extendable platform 38 of the loading system 10.

In the present invention, electrical power can be provided either by shore power or by an on-board generator 64. The generator 64 can be a diesel generator. Diesel fuel can be provided in an on-board fuel tank in communication with the generator 64. The tank may contain sufficient fuel to constantly power the generator 64 throughout several washing cycles, for example, approximately 35 gallons (135 liters) of diesel fuel.

Referring again to FIGS. 3, 4 and 5, the portable platform 36 for the apparatus may be housed within or otherwise coupled to a portable trailer 66. A skid holding the pressure washer may be affixed to the outside of the trailer 66. This arrangement has the advantage of allowing the heat produced by the pressure washer to dissipate; whereas if the pressure washer is positioned inside the trailer 66, it could create an undesirable buildup of heat inside the trailer 66. The washing fluid heater 52 may be positioned outside of the trailer 66 for the same reason. An access door 68 may be located on the trailer 66 to allow an operator to enter and exit the trailer 66. The door 68 should be of sufficient size to allow a human operator to pass through.

The extendable platform 38 may be nearly equal in length to the washing area 16, to thereby allow the maximum number of portable devices 14 to be loaded. Alternatively, in a linear feed design such as illustrated in FIGS. 3-5, portable devices 14 may be fed into the washing area 16 on a single basis, continuously, or in groups of practically any number depending on the current job requirements. The extendable platform 38 may be slightly narrower than the washing area 16 to allow space for the spray bar mechanism 42. The extendable platform 38 can be substantially automated, such that by the touch of a single button the operator causes the door 18 to open and the extendable platform 38 to extend. Once the extendable platform 38 is loaded with the desired number of portable devices 14, by the touch of a single button the operator causes the extendable platform 38 to convey the portable devices 14 into the washing area 16. The entry door or doors 18 may be positioned on the aft (rear) wall of the washing area 16 (the wall opposite the wall most proximate to the towing vehicle), and immediately at the back of the trailer 66. The trailer 66 may include a loading ramp that supports the extendable platform 38 during operation. A motor 106 that drives the extendable platform 38 may be positioned inside or outside the washing area 16.

The washing system 22 can be positioned such that the spray bar 42 is contained within the washing area 16 on a track that allows the spray bar 42 to move fore and aft, driven by a spray bar drive belt 40 positioned within the washing area 16 near the top surface of the washing area 16. An electric, hydraulic, or mechanical power take off (PTO) motor that drives the belt 40 may be positioned either inside or outside of the washing area 16. In this embodiment, the washing fluid reservoir 24 is positioned in the fore end of the trailer 66 outside of the washing area 16. One of ordinary skill in the art, in view of the present discussion, will appreciate that the belt drive motor, as well as most any of the element used in constructing the present invention, may be arranged in such a fashion as to create a system with the highest reliability and overall efficiency. The washing fluid heater 52 and pressure washer may be positioned outside of the trailer 66, attached to the surface of the trailer 66 at the fore end. The at least one radiation emitting lamp 26, according to one embodiment, is fastened to the spray bar 42 and operates as the spray bar 42 moves fore and aft.

The washing system 22 and radiation emitting lamps 26 may be substantially automated, such that by pressing a single button the operator causes the spray bar 42 to start moving, causes washing fluid to spray from the spray nozzles 44 onto the portable devices 14, causes the radiation emitting lamp 26 to activate and causes all three functions to cease after predetermined times. An indicator, for example an audio, visual or tactile mechanism, may communicate to the operator when the washing system 22 and the at least one radiation emitting lamp 26 are active and/or inactive.

The applicator 28 may be positioned on the frame of the door(s) 18 to allow the portable devices 14 to be misted during unloading. The antimicrobial substance reservoir 32 is positioned inside the trailer 66, outside of the washing area 16, near the port wall of the trailer 66. A pump and compressor for the mister 56 may be positioned proximate to the antimicrobial substance reservoir 32, and slightly aft. The applicator 28 may be substantially automated such that by the touch of a single button the operator causes the door 18 to open, the mister 56 to produce an anti-microbial mist, the extendable platform 38 holding washed portable devices 14 to extend, and the mister 56 to cease producing mist after the extendable platform 38 has fully extended.

The washing fluid collector 30 can include a sump 20 positioned at the bottom of the washing area 16. The sump pump is positioned immediately fore of the washing area 16 inside the trailer 66. The washing fluid recycling system 34 is positioned immediately fore of the washing area 16 inside the trailer 66, although elements of the washing fluid recycling system 34 are positioned throughout the fore end of the trailer 66. The washing fluid reservoir 24 is a few feet fore of the skid that contains the main body of the washing fluid recycling system 34.

The diesel tank may be in the fore port corner of the trailer 66. The diesel generator 64 is positioned in the fore starboard corner of the trailer 66. Overhead illumination may be provided throughout the trailer 66 and in the area aft of the trailer 66 where portable devices 14 will be loaded and unloaded. A control area is included inside the trailer 66 toward the aft end.

There may be sufficient space in the trailer 66 to allow the operator to move about, and for such non-essential elements as a large trash can (for example, 32 gallons) and/or a storage rack.

The trailer 66 may be towed (or otherwise transported) to a site proximate to the portable device to be sanitized. The portable device 14 is then sanitized, and the trailer 66 is then transported away from the site. Any wastewater that is produced during the washing process is disposed of away from the site proximate to the portable device.

It will be appreciated by persons of ordinary skill in the art that various embodiments of the present invention are not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A sanitizing system for sanitizing a portable device, comprising:
a transport system configured to facilitate loading and unloading of at least one portable device in a washing area, the washing area adapted to expose the at least one portable device to a washing fluid delivered while the at least one portable device is in the washing area;
at least one radiation emitting lamp positioned to expose the at least one portable device to germicidal radiation;
an applicator capable of dispensing an antimicrobial substance, the applicator positioned to place the antimicrobial substance in contact the at least one portable device; and
a washing fluid collector coupled to the washing area for collecting used washing fluid, thereby preventing used washing fluid from being released into a local environment.

2. The sanitizing system of claim 1, further comprising a portable platform, wherein the transport system, the washing area, the radiation emitting lamp, the applicator, and the washing fluid collector are supported by the portable platform.

3. The sanitizing system of claim 2, wherein the portable platform is a mobile trailer.

4. The sanitizing system of claim 2, wherein the portable platform is a truck bed.

5. The sanitizing system of claim 2, wherein the portable platform is a modular structure that is temporarily fixed in a location for operation of the sanitizing system.

6. The sanitizing system of claim 5, wherein the modular structure may be replaced by a substitute modular structure comprising the portable platform for purposes of quickly servicing the sanitizing system at the fixed location.

7. The sanitizing system of claim 1, wherein the at least one portable device comprises at least one of a shopping cart, a hand basket, a wheelchair, a walker, a meat rack, a bakery cart, and a luggage cart.

8. The sanitizing system of claim 1, wherein the loading system comprises at least one extendable platform movable between an extended position and a retracted position.

9. The sanitizing system of claim 1 further comprising a spray bar movable linearly relative to the portable device and including at least one spray nozzle for delivery of the washing fluid.

10. The sanitizing system of claim 9, wherein the at least one radiation emitting lamp is mechanically coupled to the spray bar.

11. The sanitizing system of claim 9 further comprising a pressure washer in fluid communication with the at least one spray nozzle.

12. The sanitizing system of claim 1, wherein the radiation emitting lamp is an ultraviolet radiation emitting lamp.

13. The sanitizing system of claim 1, wherein the applicator comprises a low pressure mister for dispensing the antimicrobial substance in the form of a mist onto the portable device.

14. The sanitizing system of claim 1, wherein the washing fluid collector comprises a sump.

15. The sanitizing system of claim 1 further comprising a washing fluid heater, and wherein the washing fluid comprises heated water.

16. The sanitizing system of claim 1, further comprising a washing fluid recycling system in fluid communication with the washing fluid collector.

17. The sanitizing system of claim 1, further comprising a basket tree adapted to expose the portable device to the washing fluid and the germicidal radiation, the basket tree comprising: a base; a vertical trunk having a lower end and an upper end, wherein the lower end rests on the base; at least one basket hook, the basket hook being supported by the trunk.

18. The sanitizing system of claim 1, wherein the washing area is completely enclosed when the washing system is active.

19. The sanitizing system of claim 1, wherein the transport system automatically loads and unloads the at least one portable device to/from the washing area.

20. The sanitizing system of claim 19, wherein the at least one portable device comprises a plurality of shopping carts, and wherein the transport system automatically loads and unloads the plurality of shopping carts to/from the washing area.

21. The sanitizing system of claim 19, wherein the at least one portable device comprises a plurality of shopping carts, and wherein the plurality of shopping carts have interlocked shopping carts such that a forward/rearward force applied to a rear-most/forward-most shopping cart in the plurality of shopping carts will cause the plurality of shopping carts to move forward/rearward as one moving unit, and wherein the transport system loads and unloads the plurality of shopping carts to/from the washing area as one moving unit.

22. The sanitizing system of claim 1, wherein the sanitizing system operates automatically for a pre-determined period of time.

23. The sanitizing system of claim 1, wherein the radiation emitting lamp operates automatically for a pre-determined period of time.

24. A method for sanitizing a portable device with a sanitizing system, comprising:
loading the portable device into a washing area;
exposing the portable device to a washing fluid;
irradiating the portable device with germicidal radiation;
recovering the washing fluid such that substantially all of the used washing fluid does not escape into a local environment;
treating the portable device with an antimicrobial substance; and
unloading the portable device from the washing area.

25. The method of claim 24, wherein loading the portable device into the washing area further comprises placing the portable device on an extendable platform, the extendable platform being in an extended position, and transporting the at least one portable device into and through the washing area using a linear feed system.

26. The method of claim 24, wherein recovering the washing fluid comprises collecting the washing fluid in a sump, and pumping the washing fluid to a used washing fluid reservoir or washing fluid recycling system with a sump pump.

27. The method of claim 24, further comprising the step of transporting an apparatus for sanitizing the portable device to a site proximate to the portable device.

28. The method of claim 24, further comprising the step of replacing the sanitizing system as a modular unit with a renewed sanitizing system, and transporting the sanitizing system to a remote location for at least one of servicing, renewing and recycling elements thereof necessary for reuse of the sanitizing system.

29. The method of claim 28, wherein the elements necessary for reuse of the sanitizing system comprise at least one of a washing fluid, a germicidal radiation source, and an antimicrobial substance.

30. The method of claim 29, wherein the washing fluid comprises water.

31. The method of claim 29, wherein the washing fluid contains a biodegradable detergent.

32. The method of claim 29, wherein the germicidal radiation source emits ultra-violet radiation.

33. The method of claim 29, wherein the germicidal radiation source emits non-ionizing radiation.

34. The method of claim 29, wherein the antimicrobial substance is biodegradable.

35. The method of claim 29, wherein the antimicrobial substance is applied using a mist.

36. The method of claim 29, wherein the antimicrobial substance is activated when exposed to specific radiation.

37. The method of claim 29, wherein the antimicrobial substance bonds to an applied surface of the at least one portable device when the antimicrobial substance is exposed to specific radiation.
